(19) United States
(12) Patent Application Publication
(10) Pub. No.: US 2018/0192914 A1
(43) Pub. Date: Jul. 12, 2018

(54) DETERMINING METABOLIC PARAMETERS

(71) Applicant: KONINKLIJKE PHILIPS N.V., EINDHOVEN (NL)

(72) Inventors: Alberto Giovanni Bonomi, Eindhoven (NL); Christian Andreas Tiemann, Eindhoven (NL); Wei Chen, Eindhoven (NL)

(21) Appl. No.: 15/741,000

(22) PCT Filed: Jun. 23, 2016
(86) PCT No.: PCT/IB2016/053729

§ 371 Date: Dec. 29, 2017

Related U.S. Application Data
(60) Provisional application No. 62/190,297, filed on Jul. 9, 2015.

(51) Int. Cl. A61B 5/083; A61B 5/00

(52) U.S. Cl. CPC A61B 5/0836; A61B 2560/0242; A61B 5/4866; A61B 5/0833

(57) ABSTRACT

Methods and systems for monitoring metabolic parameters of humans and other animals in an enclosed or semi-enclosed space such as a room. In these embodiments, metabolic parameters like basal metabolic rate, energy expenditure, and body composition are derived from environmental measurements of carbon dioxide (CO₂) production using context-aware processing algorithms. This information can be integrated in innovative coaching programs for weight management, fitness improvement, pregnancy management, and chronic disease management.

### FIELD OF THE INVENTION

The invention relates generally to methods and systems for determining metabolic parameters, and more specifically to the use of environmental measurements to determine metabolic parameters associated with one or more persons.

### BACKGROUND OF THE INVENTION

Metabolic measurements are usually based on indirect calorimetry using ventilated hood systems or respiration chambers. In order to obtain highly accurate measurements of respiratory gasses the test subject is typically confined in an airtight environment reproduced in a small room or around a bed. Such systems are highly obtrusive as they limit the activity and mobility of test subjects and are mostly unsuitable for home environments because of the cost of installation. These measuring systems are considered the “gold standard” for determining energy expenditure and metabolic rate at rest.

These systems determine metabolic parameters by controlling the air inhaled and exhaled by the test subject so that changes in the concentration of the subject's respiratory gasses (i.e., oxygen (O₂) and carbon dioxide (CO₂)) can be measured accurately. With measurements of O₂ dissipation and CO₂ production, energy expenditure can be determined by making assumptions concerning the substances metabolized by the test subject, such as carbohydrates, protein or fat.

Portable systems to measure O₂ consumption and CO₂ production have also been developed to determine metabolic rate during physical activity. These systems allow metabolic measurements outside of a controlled environment. However, they are still intrusive because the user typically has to breathe within a facemask or mouthpiece. Modified versions of these portable indirect calorimeters may be used to measure resting metabolic rate, provided that the user follows a controlled resting protocol while breathing within the device.

Accordingly, there is a need for methods and systems that can unobtrusively determine various metabolic parameters.

### SUMMARY OF THE INVENTION

This summary is provided to introduce a selection of concepts in a simplified form that are further described below in the Detailed Description section. This summary is not intended to identify key features or essential features of the claimed subject matter, nor is it intended to be used as an aid in determining the scope of the claimed subject matter.

Various embodiments of the invention provide methods and systems for determining metabolic parameters of humans and other animals in an enclosed or semi-enclosed space such as a room in a house without confining a subject to an airtight environment or requiring a subject to breathe in a mask or mouthpiece connected to a gas analyzer in order to obtain such measurements. Monitoring metabolic parameters of humans in a room is an attractive feature for a system providing services related to personal health and management of home spaces.

In one aspect, embodiments of the present invention relate to a method for estimating metabolic parameters. The method includes utilizing at least one item of contextual data to infer the presence of at least one person in proximity to a sensor in an interior space; obtaining at least one environmental measurement concerning the interior space from the sensor when said at least one person is present; and computing at least one metabolic parameter associated with the at least one person utilizing, at least in part, the at least one environmental measurement.

In one embodiment, the at least one contextual data is selected from the group consisting of ambient temperature, ambient noise, ambient humidity, ambient carbon dioxide, ambient oxygen, the presence of a heat source, and time of day. In one embodiment, the at least one environmental measurement is selected from the group consisting of ambient carbon dioxide and ambient oxygen. In one embodiment, the at least one metabolic parameter is selected from the group consisting of resting metabolic rate, muscle mass, body composition, and energy expenditure.

In one embodiment, utilizing contextual data to infer the presence of at least one person in proximity to a sensor comprises the application of a rule to the contextual data to decide the presence of at least one person in proximity to the sensor. In one embodiment, at least one of the contextual data and the environmental measurement is filtered. In one embodiment, the at least one environmental measure is adjusted to account for at least one factor affecting the level of the environmental measure in the indoor space, the factor selected from the group consisting of diffusion, emission, dissipation, active transport, and radiation of the environmental quantity.

In one embodiment, computing the at least one metabolic parameter comprises the conversion of the at least one environmental measurement into a volumetric measurement utilizing the characteristics of the interior space. In some embodiments, the method may further include using the rate of change of the volumetric measurement to calculate a rate of energy expenditure and the at least one metabolic parameter.

In another aspect, embodiments of the present invention relate to an apparatus for estimating metabolic parameters. The apparatus includes a computing unit in communication with a contextual data sensor to measure contextual data concerning an interior space in proximity to the contextual data sensor and a sensor which is present in the environment or as part of a wearable system to obtain at least one environmental measurement concerning the interior space. The contextual data is used to infer the presence of at least one person in proximity to the environmental sensor in an interior space. The sensor is used to obtain at least one environmental measurement concerning the interior space when at least one person is present. The computing unit is used to compute at least one metabolic parameter associated with the at least one person utilizing, at least in part, the at least one environmental measurement concerning the interior space when the at least one person is present.

In one embodiment, the contextual data is selected from the group consisting of ambient temperature, ambient noise, ambient humidity, ambient carbon dioxide, ambient oxygen, the presence of a heat source, and time of day. In one embodiment, the at least one environmental measurement is selected from the group consisting of ambient carbon dioxide and ambient oxygen. In one embodiment, the at least one metabolic parameter is selected from the group consisting of resting metabolic rate, muscle mass, body composition, and energy expenditure.

In one embodiment, utilizing contextual data to infer the presence of at least one person in proximity to the environmental sensor comprises the application of a rule to the contextual data to decide the presence of at least one person in proximity to the environmental sensor. In one embodiment, the apparatus further includes at least one filter that receives at least one of contextual data and environmental measures.

In one embodiment, computing the at least one metabolic parameter includes the conversion of the at least one environmental measurement into a volumetric measurement utilizing the characteristics of the interior space. The rate of change of the volumetric measurement may be used to calculate a rate of energy expenditure and the at least one metabolic parameter. In one embodiment, the at least one environmental measure is adjusted to account for diffusion.

In one embodiment the computing unit, the contextual data sensor, and the sensor are contained in the same apparatus. In one embodiment the computing unit, the contextual data sensor, and the sensor are distributed components that communicate with each other.

These and other features and advantages, which characterize the present non-limiting embodiments, will be apparent from a reading of the following detailed description and a review of the associated drawings. It is to be understood that both the foregoing general description and the following detailed description are explanatory only and are not restrictive of the non-limiting embodiments as claimed.

### BRIEF DESCRIPTION OF DRAWINGS

Non-limiting and non-exhaustive embodiments are described with reference to the following figures in which:

FIG. 1 is a flowchart of a method for determining metabolic parameters in accord with the present invention;

FIG. 2 is an exemplary graph of environmental data collected in accord with the present invention;

FIG. 3 is an exemplary graph showing changes in ambient carbon dioxide with time in an unmonitored space;

FIG. 4 illustrates estimates of resting metabolic rate (RMR) determined from environmental measurements in accord with one model used by embodiments of the present invention;

FIG. 5 is a histogram of energy expenditure estimations for a plurality of persons developed using a second model used by embodiments of the present invention; and

FIG. 6 is a block diagram of an exemplary apparatus for determining metabolic parameters in accord with the present invention.

In the drawings, like reference characters generally refer to corresponding parts throughout the different views. The drawings are not necessarily to scale, emphasis instead being placed on the principles and concepts of operation.

### DETAILED DESCRIPTION OF THE INVENTION

Various embodiments are described more fully below with reference to the accompanying drawings, which form a part hereof, and which show specific exemplary embodiments. However, embodiments may be implemented in many different forms and should not be construed as limited to the embodiments set forth herein; rather, these embodiments are provided so that this disclosure will be thorough and complete, and will fully convey the scope of the embodiments to those skilled in the art. Embodiments may be practiced as methods, systems or devices. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one embodiment of the invention. The appearances of the phrase “in one embodiment” in various places in the specification are not necessarily all referring to the same embodiment.

Some portions of the description that follow are presented in terms of symbolic representations of operations on non-transient signals stored within a computer memory. These descriptions and representations are the means used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices.

Certain aspects of the present invention include process steps and instructions that could be embodied in software, firmware or hardware, and when embodied in software, could be downloaded to reside on and be operated from different platforms used by a variety of operating systems.

The present invention also relates to an apparatus for performing the operations herein. This apparatus may be specially constructed for the required purposes, or it may comprise a general purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

The processes and displays presented herein are not inherently related to any particular computer or other apparatus. Various general purpose systems may also be used with programs in accordance with the teachings herein, or it may prove convenient to construct more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein, and any references below to specific languages are provided for disclosure of enablement and best mode of the present invention.

In addition, the language used in the specification has been principally selected for readability and instructional purposes, and may not have been selected to delineate or circumscribe the inventive subject matter. Accordingly, the disclosure of the present invention is intended to be illustrative, but not limiting, of the scope of the invention, which is set forth in the claims.

In brief overview, various embodiments of the invention provide methods and systems for monitoring metabolic parameters of humans and other animals in an enclosed or semi-enclosed space such as a room. In these embodiments, metabolic parameters like basal metabolic rate, energy expenditure, and body composition are derived from environmental measurements of carbon dioxide (CO₂) production using context-aware processing algorithms. This information can be integrated in innovative coaching programs for weight management, fitness improvement, pregnancy management, and chronic disease management.

Embodiments of the present invention utilize environmental sensors to quantify various relevant environmental factors in an enclosed or semi-enclosed space such as noise level, CO₂ level, room temperature, barometric pressure, and humidity. The environmental sensors may be dedicated elements contained within the embodiments or they may be components of external systems (e.g., atmospheric sensors, air purifiers, home weather stations, etc.) that may be utilized and communicated with by various embodiments. Leveraging pre-existing external systems permits embodiments of the present invention to determine metabolic parameters in an unobtrusive manner.

The environmental measurements are adjusted to account for various factors using contextual data such as the presence, number, and activity of the people under observation, the transport or diffusion of various environmental factors (e.g., CO₂ diffusion), and the nature of the environment (an enclosed or semi-enclosed space, etc.) and then used to assess metabolic features such as resting metabolic rate, total energy expenditure, diet-induced energy expenditure, and activity energy expenditure. These adjustments may be made by a dedicated processing unit such as a computing hub or any dedicated or special-purpose computer capable of these computations, such as a smartphone, phablet, desktop computer, smart appliance, etc.

In particular, contextual data may be used to identify when (i.e., what time(s) of day) the environmental measurements are particularly reliable for the assessment of metabolic parameters from CO₂ measurements, improving measurement accuracy without requiring a controlled and obtrusive environment.

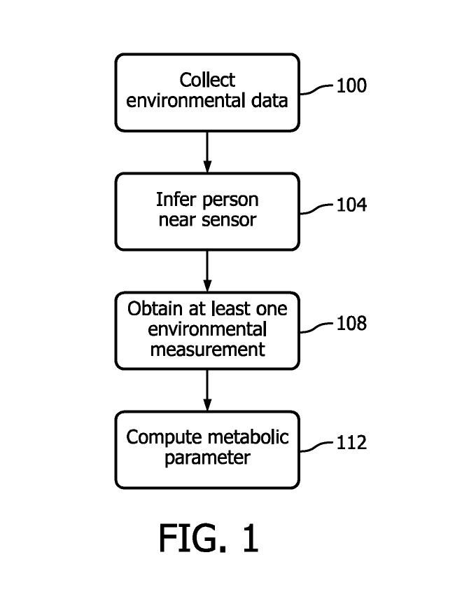

FIG. 1 presents a flowchart of an embodiment of a method to determine metabolic parameters in accord with the present invention. The method derives the metabolic parameters of one or more persons present in a room environment by combining heterogeneous sensor data with high-level contextual information and applying time series processing algorithms.

In this embodiment, a computing unit collects environmental data (i.e., information concerning the characteristics of an enclosed or semi-enclosed interior space such as a room in a house) over a period of time that may range from as little from 5-10 minutes to one or more months (Step 100). Such environmental data can be acquired using one or more multi-sensor systems or a plurality of distributed sensors and may include temperature, atmospheric pressure, humidity, room size, noise level, and/or the concentration of carbon dioxide or other ambient gases that may be relevant to determining metabolic processes or specific contextual scenarios occurring in the space. The environmental data can be analyzed using a variety of computing units to extract relevant features from the time series values and/or to assess the condition of or the context in which measurements have been collected.

A context awareness element of the invention analyzes and interprets at least a subset of the collected environmental data to develop at least one inference concerning the condition of the interior space, for example, whether the room is empty or whether there are one or more persons in the space, preferably in proximity to a sensor in that interior space (Step 104). Other possible inferences concern the reliability of the environmental data, whether a plurality of subjects are present in the interior space, whether the subject(s) is engaged in activity, and the nature of the activity that the subject(s) is engaged in.

In one embodiment, these inferences are drawn by analyzing features of the collected environmental data as well as exogenous information such as, e.g., time of day or various weather conditions. This analysis may include, but is not limited to, smoothing or filtering the data in the time or frequency domain(s), computing first or second order derivatives of the data (and/or smoothing those derivatives), computing drop ratios, etc.

In another embodiment, these inferences are drawn by applying predetermined rules to the collected environmental data to see if any of these rules are satisfied. The rules themselves may be determined manually or automatically (e.g., using data clustering) according to collected training data and prior knowledge.

The following is one example of an algorithm that determines contextual information (i.e., whether a room is occupied and, if so, the approximate number of occupants) from environmental data and also determines whether the data is reliable for metabolic measurements. Threshold1 and Threshold2 represent the quantity of CO₂ production for a single person relative to a percentage of an average resting metabolic rate value (50% and 150% of 1600 kcal/day, respectively):

```
If NoiseLevel > 40 dB
      If CO2 consecutive quotient > 100%
            Presence = TRUE
      Else
      Presence = FALSE
End
If CO2 > Threshold-P1
      If CO2 < Threshold-P2
      Occupancy = 1
      Else
      Occupancy > 1
      End
End
If Presence = TRUE and Occupancy = 1
      If DayTime < 11am
            RMR_Reliability = TRUE
End
```

As is apparent to one of ordinary skill, the exemplary algorithm first considers the noise level in the room and the trend in carbon dioxide measurements to decide whether the space is occupied at all. When the noise level exceeds 40 dB and the consecutive quotient in CO₂ exceeds 100% the algorithm decides there are people present in the room. If the CO₂ consecutive quotient is decreasing the algorithm indicates the absence of people in the room.

The algorithm then considers the level of carbon dioxide present to decide how many individuals are present in the room, i.e., if the level of carbon dioxide exceeds more than 150% of what would be expected from a single individual with an average resting metabolic rate, then the algorithm assumes there are multiple people present in the space. Any increase in CO₂ concentration which is below a predefined threshold is discarded as indicative of inaccurate measurements.

Having concluded from the environmental data that at least one subject is in proximity to an environmental sensor, then a measurement from that environmental sensor is taken (Step 112) and used to determine at least one metabolic parameter for the at least one subject (Step 116).

After establishing one or more items of contextual data such as presence in a space, number of occupants in a space, type of activity occurring in a space, etc. (Step 104), derived from the collection of environmental data (Step 100), an environmental measurement (e.g., a measurement of ambient CO₂; Step 108) is used to determine one or more metabolic parameters (Step 112).

In the calculation phase (Step 112) the environmental measurements (e.g., CO₂ measurements) may be converted from standard quantities related to concentration (e.g., parts per million [ppm]) into volumetric measurements (e.g., mL) to facilitate further computations. The conversion process may utilize information on the size of the space or environment where the measurements are collected, which can be manually entered by a user or detected automatically using, e.g., systems based on optical sensors or cameras.

The rate of CO₂ production (Step 112) may be determined from the environmental measurement (Step 108) utilizing previously gathered environmental measurements (Step 100) and/or inferred contextual information (Step 104) related to the duration of occupation of the space. For example, an algorithm can use a change in a previously-detected feature or a previously-applied rule to determine when the space becomes occupied or unoccupied, and thereby determine the duration of occupation. In one embodiment, an increase in CO₂ concentration can be used to identify a start time for the occupation and a drop in CO₂ concentration may be used to determine that the space is vacant, that the number of occupants in the space has changed, or that ventilation has changed, thereby identifying the endpoint of the occupation and its overall duration.

The overall duration and the CO₂ measurements can be applied to the following equation to compute the rate of CO₂ production (VCO₂):

$${VCO}_{2} [ ml \text{/} min ] = \frac{{CO}_{2} end [ ml ] - {CO}_{2} start [ ml ]}{duration [ min ]} \qquad ( Eq . 1 )$$

With an estimate of VCO₂, a respiratory quotient value (RQ) may be assumed to permit the calculation of energy expended according to published equations such as the Weir equation (Step 112). For example, the RQ can be assumed to be equal to 0.8 unless sustained activities are being carried out by a monitored user in the space. The Weir equation is described in “New methods for calculating metabolic rate with special reference to protein metabolism,” in The Journal of Physiology, vol. 109, no. 1-2, pp. 1-9 (1949), the entire contents of which are hereby incorporated by reference as if set forth in their entirety herein.

With a value computed for energy expended, various metabolic parameters can be determined for a monitored user (Step 112). For example, if context data suggests that the user just woke up (e.g., the time is between 6 a.m. and 8 a.m.), then the energy expenditure data can be used to compute resting metabolic rate (RMR). If context data suggests that the user has just eaten (e.g., the time is between 11 a.m. or 1 p.m., or the CO₂ level suggests that a gas stove has been operated), then the energy expenditure data may be used to compute diet-induced thermogenesis (DIT). If context data suggests that a user has been performing a particular activity (e.g., the sound data suggests that the user has been operating a treadmill), then the energy expenditure data can be used to calculate the energy cost of a specific activity (AEE).

With a value for RMR, estimates of body composition, muscle mass and fat mass can be derived using appropriate prediction equations provided by scientific literature. Such equations may be found in K. M. Nelson, R. L. Weinsier, C. L. Long, and Y. Schutz, “Prediction of resting energy expenditure from fat-free mass and fat mass,” Am. J. Clin. Nutr., 56:848-56 (1992), the entire contents of which are hereby incorporated by reference as if set forth in their entirety herein. To improve the estimation accuracy, subsequent assessments of RMR, DIT or AEE may be filtered or provided as input to an estimator to update the estimates of these metabolic variables.

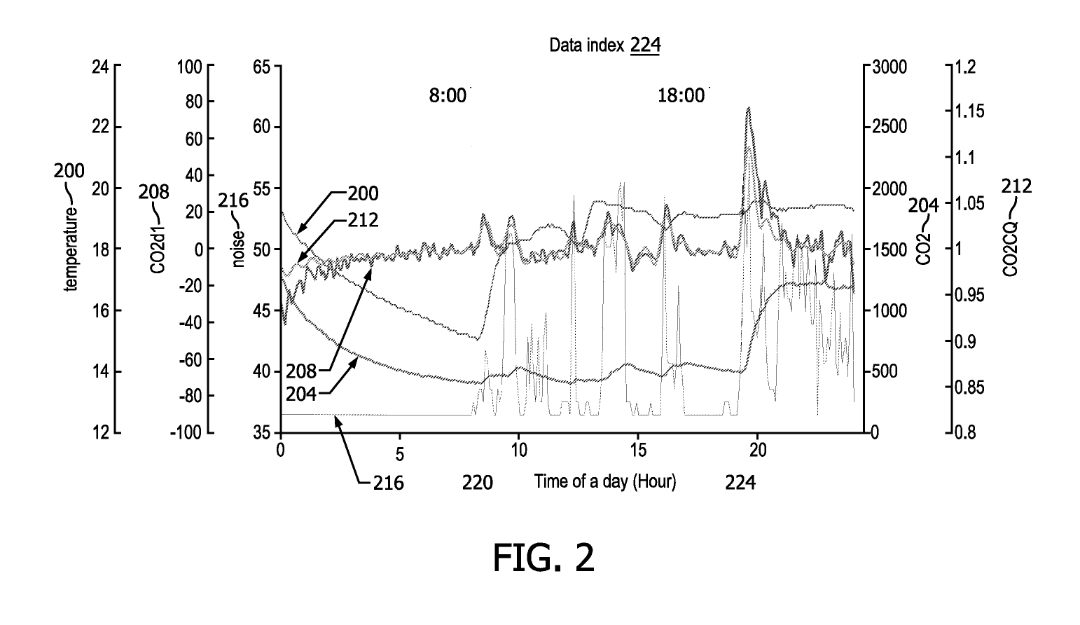

By combining information on noise level, changes in CO₂ concentration and the time of day several user activities could be identified, for example, resting in the morning, having breakfast, having lunch, having dinner, sleeping, napping, watching TV, etc. Towards that end, FIG. 2 presents an exemplary graph of various time-series values of environmental data collected every five minutes by one embodiment of the present invention in a semi-enclosed space approximately 8 m×3.5 m×3 m. As shown by the x-axis, the time-series values are collected over a single 24-hour period. The time-series values include ambient temperature 200 in the space, ambient carbon dioxide 204 in the space, rate of change of the ambient carbon dioxide (i.e., first derivative) 208, consecutive quotients of ambient carbon dioxide 212, and ambient noise 216 in the space.

As is evident from the plot of the time-series data, the changes in these data series are associated with various events that involve a change in the occupancy of the space and/or the activity level of the occupants of the space. For example, a rise in the ambient temperature 200 shortly after 8 a.m. is associated with the engagement of the space's heating system 220. The ambient noise 216 indicates that the space is virtually silent while the occupants are asleep, from midnight through 8 a.m., and that the level of noise varies through the day with various activities. Similarly, the level of ambient carbon dioxide 204 declines overnight while the occupants are asleep, and then rises throughout the day, with a notable rise in the evening as the occupants engage various kitchen appliances 224 to prepare the evening meal.

In some embodiments, contextual awareness may be substituted or augmented by user input. Information on a specific activity being performed in the monitored space can be provided by a user, as well as information on the status of the environment such as whether doors and windows are open or closed. Furthermore, context classification may be achieved using clustering techniques and data-driven rules to associate data with groups having particular characteristics linked to contextual scenarios.

In some embodiments, the measurements of CO₂ concentration (Step 108) are corrected by the amount of CO₂ expected to be flowing out of the monitored space due to openings in doors and windows that may be present. Such correction improves the quality of the measurements of CO₂ resulting from metabolic processes. An estimate of the rate of CO₂ decrease due to such diffusion can be empirically obtained from the collected data (Step 100) during a period when people are absent from the monitored space by determining fitting functions (e.g., linear, exponential, etc.) that model the decrease in CO₂ concentration with time. This method allows tailoring the correction factor to the specific characteristics of the monitored space.

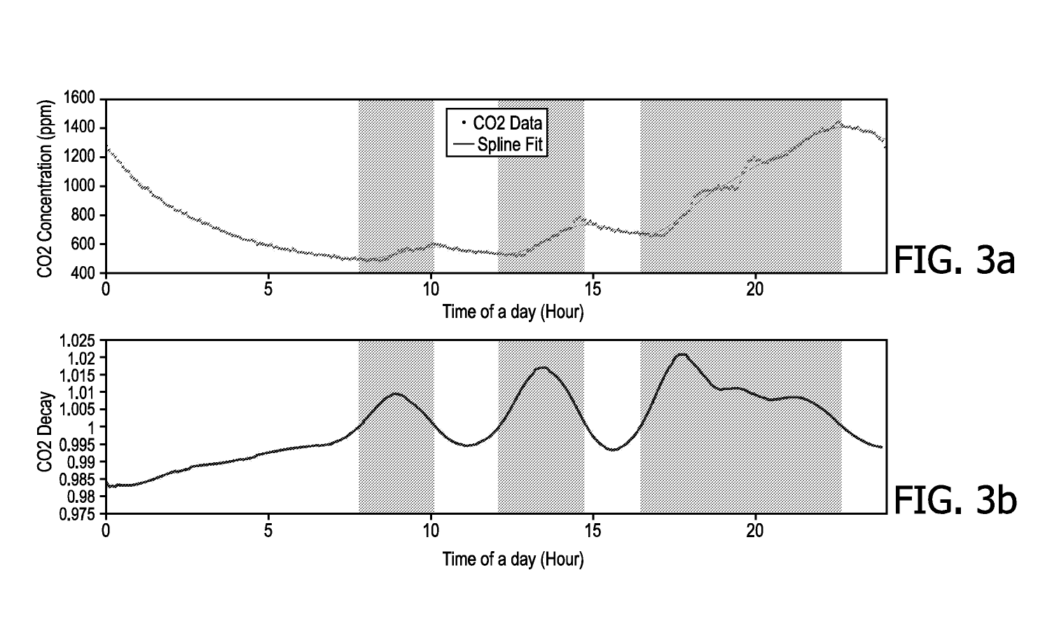

By way of example, FIG. 3 depicts a graph showing changes in ambient carbon dioxide with time in an unoccupied monitored space. The grey areas indicate the periods in which the monitored space was occupied; this can be determined through monitoring ambient sound, a pyrometer, a motion sensor, the assumption that an increasing CO₂ concentration is indicative of occupation, etc. FIG. 3(a) shows the individual CO₂ concentration measurements as well as a spline fit to the measurement data. FIG. 3(b) shows the rate of change in carbon dioxide concentration as a function of time, which can then be applied to ambient carbon dioxide measurements as discussed above. In this particular example, a value of 99.1% of CO₂ concentration decay could be used in this environment to correct for gas flowing out of the room.

Other embodiments of the present invention adopt a different approach to CO₂ diffusion modeling and correction. In these embodiments, a processing unit is used that has access to environmental measurement data collected over time. One or more sensors are used that measure the environmental CO₂ as frequently as possible, preferably every five minutes or even more often. An algorithm estimates the CO₂ exhalation rate, which is subsequently used to calculate a user's energy expenditure.

A first CO₂ sensor (Sensor A) is located inside a room, e.g., a living room or office. The environmental CO₂ in this room is influenced by the exhalation of CO₂ by the inhabitants of the room. Multiple CO₂ sensors may be used, with each sensor placed in a different room.

Optionally, an additional CO₂ sensor (Sensor B) is placed outside the building containing the room. An outdoor sensor is not required, but it will measurably improve the accuracy of the estimated energy expenditure. The outdoor sensor measures the outdoor CO₂ which serves as a baseline to be used to estimate the net diffusion of CO₂ from the room to the outside world.

In addition, environmental sensors can be used to measure the time and the sound level, temperature, pressure, and humidity in the room. These measurements can provide context to the estimated energy expenditure, for instance, to determine whether the person is resting, active, or sleeping, as discussed above.

The computation of the estimate in accord with these embodiments begins with selecting a subset of the gathered data during a desired period of time. This can be done by a user or in an automated way by, e.g., selecting data from the last x weeks or selecting data previously unprocessed data.

The first and second derivatives of the room CO₂ signal (i.e., collected by Sensor A) are used to select temporal subsets of the collected room CO₂ data where the room CO₂ concentration increases or decreases. The first and second derivatives may be calculated using a processed variant of the CO₂ signal that has been, e.g., filtered for noise. For example, when the first derivative is positive for a consecutive period of at least 20 minutes, the subset is considered as an increasing CO₂ period. Likewise, when the first derivative is negative for a consecutive period of at least 20 minutes, the region is considered as a decreasing CO₂ period.

The second derivative is used to fine-tune the start time of the increasing and decreasing periods. For increasing CO₂ periods, the time point where the second derivative is maximal is used as the start point. Likewise, for decreasing CO₂ periods the time point where the second derivative is minimal is used as the start point. This additional step may be used to select temporal subsets of the collected room CO₂ data where changes in CO₂ are most prominent, e.g., most likely due to human behavior and to omit onset periods where changes in CO₂ are still small. Contextual data about temperature, humidity, pressure, and sound level can also be used to omit periods where the energy expenditure cannot be estimated accurately, e.g., during cooking, as discussed above.

Having identified periods of increasing and decreasing CO2 concentration in the time periods of interest, a computational model is used to simulate and reproduce the dynamics observed during the increasing and decreasing periods. In one embodiment, the model consists of two parts: a first additive part that models factors adding CO₂ to the environment (e.g., human exhalation), and a second subtractive part that models factors removing CO₂ from the environment (e.g., via diffusion/transport of CO₂ to adjacent areas).

In one embodiment, the computational model is an ordinary differential equation (ODE) that models the change in CO₂ concentration over time:

$$\frac{d [ {CO}_{2} ] ( t )}{dt} = {c}_{1} - {c}_{2} ( [ {CO}_{2} ] ( t ) - [ {CO}_{2}^{out} ] ( t ) ) \qquad ( Eq . 2 )$$

Here, parameter c₁ represents the additive factors (i.e., human CO₂ excretion) and parameter c₂ represents the subtractive factors (i.e., the diffusion constant). As described by Eq. 2, the diffusion rate at time t is given by the diffusion constant multiplied by the difference between the indoor CO₂ concentration ([CO₂](t), as measured by Sensor A) and the outdoor CO₂ concentration ([CO₂^{out}](t), as measured by Sensor B) at that point in time.

In embodiments that do not employ Sensor B, an estimate of the outdoor CO₂ concentration can be used. Parameters c₁ and c₂ are initially unknown and may be estimated using a least squares optimization technique that determines the parameters that minimize a difference measure (e.g., the sum of squared differences) between the simulated and measured CO₂ profile. The optimization procedure is performed for all identified increasing and decreasing periods. Hence, a vector ĉ of ĉ₁ and ĉ₂ estimations is obtained, i.e.,

$$\hat{c} = [ \begin{matrix}{\hat{c}}_{1} \\ {\hat{c}}_{2}\end{matrix} ] \text{:}$$

ĉ=arg min_{c}(Σᵢ₌₁^{N}([CO₂^{data}](tᵢ)−[CO₂^{simulation}](tᵢ,c))²) (Eq. 3)

The accuracy of an inferred parameter can be assessed by determining confidence bounds of the estimation. A bootstrapping approach can, for instance, be employed for this purpose by repeating the parameter estimation for different realizations of the data, resulting in a distribution of estimations. Subsequently, confidence intervals can be determined from the resulting distribution of estimations. Different data realizations may be obtained by adding different randomly-sampled noise realizations to the data. The information can, for instance, be used to avoid inaccurate estimations or to weight multiple estimations obtained during a certain time period.

Each estimated parameter ĉ₁ may be used to obtain a corresponding energy expenditure value. First, ĉ₁, which is a concentration flux [ppm CO₂/min], is converted to a volume flux (VCO₂) expressed as [mL CO₂/min] using information about room size:

VCO₂=ĉ₁Vᵣₒₒₘ (Eq. 4)

where Vᵣₒₒₘ is the room volume in m³. The room volume can be provided to the system manually or can be determined automatically using, e.g., systems based on optical sensors and cameras, ultrasonics, etc. Subsequently, the aforementioned Weir equation is used to calculate the user's energy expenditure (EE):

$$EE = 1.44 ( 3.9 \frac{{VCO}_{2}}{RQ} + 1.1 {VCO}_{2} ) \qquad ( Eq . 5 )$$

where RQ is the respiratory quotient, which is often assumed to be around 0.82 during resting conditions. One of ordinary skill will recognize, of course, that the specific coefficient values of the model may vary among various embodiments of the invention and are otherwise non-limiting.

The energy expenditure is calculated for each estimated parameter ĉ₁, resulting in a vector of EE values. The histogram of such a vector could reveal different modes of EE values corresponding to different persons or groups of persons. Clustering techniques can be used to extract the different modes. This makes it possible to track the energy expenditure of different individuals over time. Information about energy expenditure during different activities of daily living can be integrated in innovative coaching programs for personalized weight management, fitness improvement, pregnancy management, and chronic disease management.

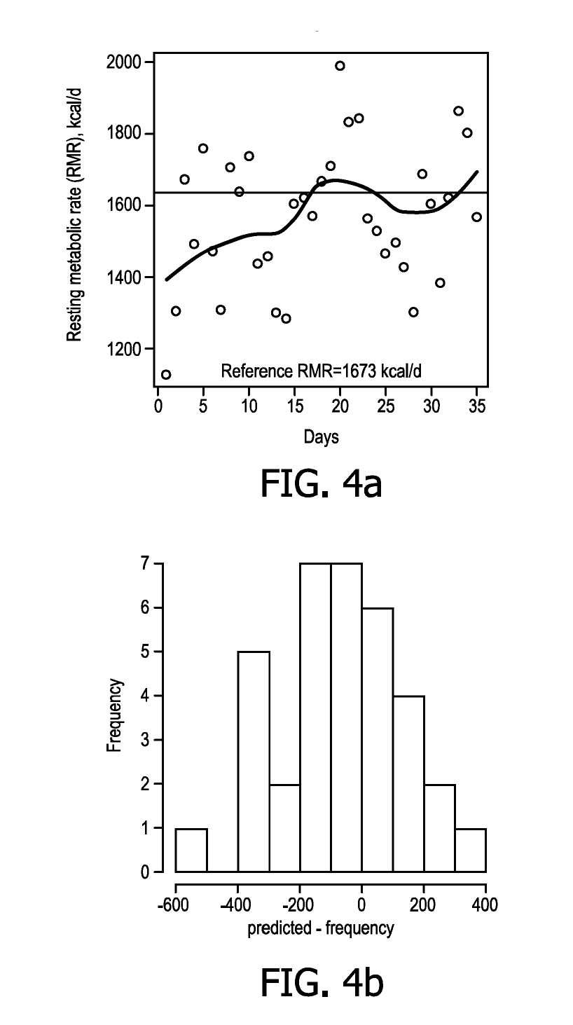

FIG. 4 illustrates estimates of RMR for a single individual determined from environmental measurements utilizing the inventive methods and apparatus discussed above in connection with FIG. 1. To be specific, the RMR was determined by using context identification to identify the presence of people in the monitored space, detecting a singular occupant, automatic detection of the departure of occupants, detection of occupant presence in the morning, correction for CO₂ diffusion and averaging the daily RMR estimate with computed RMR values from the previous days. The line in FIG. 4(a) represents a running average for the global assessment of RMR for each monitoring day which shows convergence to the reference RMR.

As depicted in FIG. 4(a), initial estimates may be inaccurate before eventually converging on the true value. As shown, when this method was applied to environmental data captured for a subject over subsequent days (over 30 days) the average bias in the estimation of RMR was below 60 kcal/day (i.e., <3%). FIG. 4(b) is a histogram of the day-by-day error in the estimate of RMR.

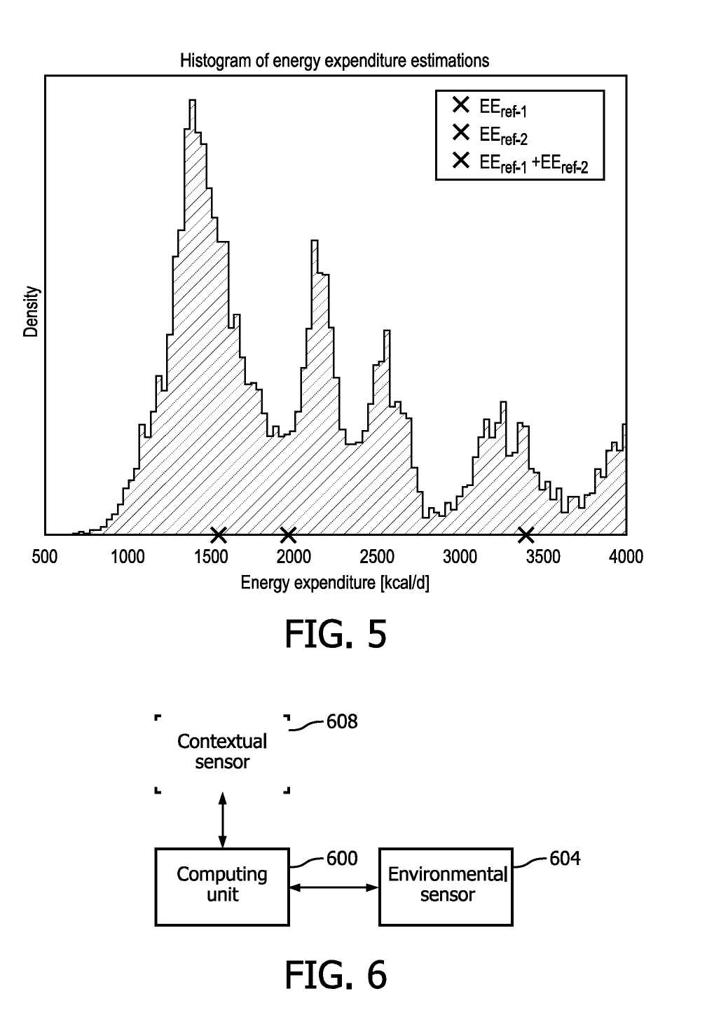

FIG. 5 presents an example of a histogram of one week's worth of energy expenditure (EE) values calculated for estimated parameters ĉ₁ using Eqs. 2-5. As is evident from the histogram, the histogram of such a vector can reveal different modes of EE values corresponding to different persons or groups of persons. Clustering techniques can then be used to extract the different modes. This provides the possibility to track the energy expenditure of individual persons over time.

In this case, the space is predominantly occupied by two persons. The crosses represent energy expenditure values based on the Harris-Benedict equation. The two modes coincide with the two persons that occupy the space (EE_{ref-1} and EE_{ref-2}), and one mode coincides with the sum of both persons (EE_{ref-1}+EE_{ref-2}). FIG. 6 presents an example of an apparatus for estimating metabolic parameters in accord with the present invention. A computing unit 600 is in communication with at least one environmental sensor 604 and, optionally, a contextual sensor 608.

The computing unit 600 may take a variety of forms, such as a local desktop or laptop computer, a set top box, an app executing on a smartphone, a tablet, a “next unit of computing” (NUC), a wireless speaker, or a remote server computer in communication with one or more of the foregoing devices, etc., but regardless of particular configuration includes sufficient computing capacity to execute the methods described above.

A variety of environmental sensors 604 may also be used in accord with the present invention, such as a microphone, a video camera, a carbon dioxide sensor, a thermometer, a pyrometer, a motion sensor, a barometer, a humidity sensor, etc. The environmental sensor 604 may take a variety of configurations and may, in some embodiments, be integrated into the computing unit 600 or be a discrete, standalone item. Notably, environmental sensors 604 provide measurements of environmental factors in a monitored space.

A variety of contextual sensors 608 may be employed in various embodiments. The contextual sensors 608 may take a variety of configurations and may, in some embodiments, be integrated into the computing unit 600 or be a discrete, standalone item. The types of environmental sensors 604 discussed above may also be employed as contextual sensors 608. Some embodiments will lack an explicit contextual sensor 608 and will instead use a single device (e.g., a CO₂ sensor) as both an environmental sensor 604 and a contextual sensor 608.

In various embodiments, the components of the apparatus are integrated into a single embodiment or housing. In other embodiments, the components are distributed through the space and communicate through wired (e.g., Ethernet, Token Ring, etc.) or wireless interconnections (e.g., 802.11x, Bluetooth, Bluetooth LE, Zigbee, etc.).

When the components are distributed, they may themselves be components of other appliances. For example, in some embodiments the computing unit 600 communicates with an environmental sensor 604 and/or a contextual sensor 608 that is part of a weather station, an air purifier, a cellphone, etc. In these embodiments, the components may be supplied by the same manufacturer or they may be supplied by different manufacturers and communicate using, e.g., a common protocol.

One such protocol would permit individual components to interoperate with each other by announcing their capabilities to each other and permitting the components to query each other for information or actions relating to their announced capabilities. In one such exemplary embodiment, a computing unit 600′, e.g., a smartphone running an app, an environmental sensor 604′, e.g., a CO₂ sensor, and a contextual sensor 608′, e.g., a pyrometer, are all supplied by different manufacturers and establish communications with each other using a personal-area network (PAN) in a home environment utilizing Bluetooth LE. Once communications are established, the environmental sensor 604′ announces that it will provide a measurement of CO₂ when queried and the context sensor 608′ announces that it will provide an indication of a person in proximity to the context sensor 608′ when queried. The computing unit 600′ operating as discussed above queries the context sensor 608′ and receives a message indicating that there is a person in proximity to the context sensor 608′. The computing unit 600′ issues a plurality of queries to the environmental sensor 604′ to obtain measurements of ambient carbon dioxide concentration at various points in time. The computing unit 600′ selects measurements that coincide with a period of occupancy by a single person and uses those measurements to compute VCO₂ and various metabolic parameters for the person under observation as discussed above.

The placement of the individual components may be apparent to an ordinary observer, such as when they are embedded in individual appliances, but they may also be concealed from ordinary view, such as when the component is embedded in a device that is ostensibly unrelated to environmental monitoring, such as a television, light bulb, or a smartphone.

Information on energy expenditure, resting metabolic rate, or related factors such as body composition and muscle mass can be integrated in innovative coaching programs for weight management, fitness improvement, pregnancy management and chronic disease management. Coaching services may use metabolic data to personalize and enhance the physiological response to a specific intervention program so to maximize the desired health benefit.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

The description and illustration of one or more embodiments provided in this application are not intended to limit or restrict the scope of the present disclosure as claimed in any way. The embodiments, examples, and details provided in this application are considered sufficient to convey possession and enable others to make and use the best mode of the claimed embodiments. The claimed embodiments should not be construed as being limited to any embodiment, example, or detail provided in this application. Regardless of whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternate embodiments falling within the spirit of the broader aspects of the general inventive concept embodied in this application that do not depart from the broader scope of the claimed embodiments.

1. A method for estimating metabolic parameters of at least one person, the method comprising:
(a) utilizing at least one item of contextual data to infer the presence of at least one person in an interior space;
(b) obtaining at least one environmental measurement concerning the interior space from the sensor when said at least one person is present; and
(c) computing at least one metabolic parameter associated with the at least one person utilizing, at least in part, the at least one environmental measurement.

2. The method of claim 1 wherein the at least one contextual data is selected from the group consisting of ambient temperature, ambient noise, ambient humidity, ambient carbon dioxide, ambient oxygen, the presence of a heat source, and time of day.

3. The method of claim 1 wherein the at least one environmental measurement is selected from the group consisting of ambient carbon dioxide and ambient oxygen.

4. The method of claim 1 wherein the at least one metabolic parameter is selected from the group consisting of energy intake, resting metabolic rate, muscle mass, body composition, and energy expenditure.

5. The method of claim 1 wherein utilizing contextual data to infer the presence of at least one person in proximity to a sensor comprises the application of a rule to the contextual data to decide the presence of at least one person in proximity to the sensor.

6. The method of claim 1 wherein at least one of the contextual data and the environmental measurement is filtered.

7. The method of claim 1 wherein computing the at least one metabolic parameter comprises the conversion of the at least one environmental measurement into a volumetric measurement utilizing the characteristics of the interior space.

8. The method of claim 7 further comprising using the rate of change of the volumetric measurement to calculate a rate of energy expenditure and the at least one metabolic parameter.

9. The method of claim 1 wherein the at least one environmental measure is adjusted to account for at least one factor affecting the level of the environmental measure in the indoor space, the factor selected from the group consisting of diffusion, emission, dissipation, and active transport of the environmental quantity.

10. An apparatus for estimating metabolic parameters of at least one person, the apparatus comprising:
a computing unit in communication with:
a contextual data sensor to measure contextual data concerning an interior space in proximity to the contextual data sensor; and
a sensor which is present in the environment or as part of a wearable system to obtain at least one environmental measurement concerning the interior space,
wherein the computing unit is configured to:
infer the presence of at least one person in proximity to the environmental sensor in an interior space based on the contextual data,
obtain at least one environmental measurement concerning the interior space via the sensor, and
compute at least one metabolic parameter associated with the at least one person utilizing, at least in part, the at least one environmental measurement concerning the interior space when the at least one person is present.

11. The apparatus of claim 10 wherein the contextual data is selected from the group consisting of ambient temperature, ambient noise, ambient humidity, ambient carbon dioxide, ambient oxygen, the presence of a heat source, and time of day.

12. The apparatus of claim 10 wherein the at least one environmental measurement is selected from the group consisting of ambient carbon dioxide and ambient oxygen.

13. The apparatus of claim 10 wherein the at least one metabolic parameter is selected from the group consisting of resting metabolic rate, muscle mass, body composition, and energy expenditure.

14. The apparatus of claim 10 wherein utilizing contextual data to infer the presence of at least one person in proximity to the environmental sensor comprises the application of a rule to the contextual data to decide the presence of at least one person in proximity to the environmental sensor.

15. The apparatus of claim 10 further comprising at least one filter that receives at least one of contextual data and environmental measurements.

16. The apparatus of claim 10 wherein computing the at least one metabolic parameter comprises the conversion of the at least one environmental measurement into a volumetric measurement utilizing the characteristics of the interior space.

17. The apparatus of claim 16 wherein the rate of change of the volumetric measurement is used to calculate a rate of energy expenditure and the at least one metabolic parameter.

18. The apparatus of claim 10 wherein the at least one environmental measure is adjusted to account for diffusion.

19. The apparatus of claim 10 wherein the computing unit, the contextual data sensor, and the sensor are contained in the same apparatus.

20. (canceled)

21. A non-transitory machine-readable storage medium encoded with instructions for execution by a processor for estimating metabolic parameters of at least one person, the non-transitory machine-readable storage medium comprising:
instructions for utilizing at least one item of contextual data to infer the presence of at least one person an interior space;
instructions for obtaining at least one enviromental measurement concerning the interior space from the sensor when said at least one person is present; and
instructions for computing at least one metabolic parameter associated with the at least one person utilizing, at least in part, the at least one environmental measurement.
